(19) United States
(12) Patent Application Publication
(10) Pub. No.: US 2016/0071293 A1
(43) Pub. Date: Mar. 10, 2016

(54) ARTIFACT-REDUCTION FOR X-RAY IMAGE RECONSTRUCTION USING A GEOMETRY-MATCHED COORDINATE GRID

(71) Applicant: KONINKLIJKE PHILIPS N.V., Eindhoven (NL)

(72) Inventors: Hanno Heyke HOMANN, Hannover (DE); Klaus ERHARD, Hamburg (DE); Tim NIELSEN, Hamburg (DE)

(21) Appl. No.: 14/890,179

(22) PCT Filed: May 14, 2014
(86) PCT No.: PCT/EP2014/059806

§ 371 Date: Nov. 10, 2015

(30) Foreign Application Priority Data
May 14, 2013 (EP) 13305606.9

(51) Int. Cl. G06T 11/00; A61B 6/00; A61B 6/02; G06T 7/00; G06T 15/08

(52) U.S. Cl. CPC G06T 11/008; G06T 7/0012; G06T 15/08; A61B 6/025; A61B 6/4085; A61B 6/461; A61B 6/54; G06T 2200/04; G06T 2207/10116; G06T 2207/10072; G06T 2207/30004; G06T 2210/41; G06T 2211/421

(57) ABSTRACT

A method for processing image data of an X-ray device (10) comprises the steps of: receiving a plurality of two-dimensional projection images (32) from an object of interest (22), wherein the projection images have been acquired by transmitting X-rays (20) through the object of interest (20) with respect to different view angles; generating a three- dimensional raw image volume (36) from the plurality of two-dimensional projection images (32) with respect to a coordinate grid (50) adapted to the geometry of the transmitted X-rays (20); and generating a deconvolved three-dimensional image (40) by applying a two- dimensional deconvolution to slices (52) of the three-dimensional raw image volume (36), which slices (32) are adapted to the coordinate grid (50).

### FIELD OF THE INVENTION

The invention relates to a method, a computer program and a computer-readable medium for processing image data of an X-ray device as well as to an X-ray device.

### BACKGROUND OF THE INVENTION

X-ray tomosynthesis is an emerging modality in many clinical applications, exhibiting e.g. better visualization of micro-calcifications and lesions in mammographic imaging than the conventional projection views.

X-ray tomosynthesis may be seen as a special kind of X-ray imaging technique, in which for an object of interest, for example a breast, a limited number of projection images from different view angles within a limited view angle range is acquired. From this, a three-dimensional image is then calculated. However, the limited view angle range may result in a poor z-resolution. The method is hence often referred to as a “2+½ dimensional” rather than as a full three-dimensional imaging technique.

For example, WO 2012 001 572 A1 shows a tomosynthesis system.

A broad range of image reconstruction techniques, including Filter-Back-Projection (FBP) or even more sophisticated iterative and statistical methods, have already been proposed. However, in general, these methods are subject to artifacts from the limited-angle system geometry. Two-dimensional deconvolution has been proposed in the field of computer tomography, but more than 25 years ago, see for example A.“P. Dhawan, R. M. Rangayyan, and R. Gordon: Wiener filtering for deconvolution of geometric artifacts in limited-view image reconstruction. Proc. SPIE 515, 168-172 (1984)”. However, the progress in other methods for suppression geometric artifacts in computer tomography was such that deconvolution methods have not been pursued since then.

### SUMMARY OF THE INVENTION

There may be a need to generate tomosynthesis images with fewer artifacts, higher contrast and better depth of field. There also may be a need to generate such images with only less computing power.

These needs are met by the subject-matter of the independent claims. Further exemplary embodiments are evident from the dependent claims and the following description.

An aspect of the invention relates to a method for processing image data of an X-ray device. Further aspects of the invention are a computer program that is adapted for performing the method, when run on a processor, and a computer-readable medium, on which such a program is stored.

According to an embodiment of the invention, the method comprises the steps of: receiving a plurality of two-dimensional projection images from an object of interest, wherein the projection images have been acquired by transmitting X-rays through the object of interest with respect to different view angles; generating a three-dimensional raw image volume from the plurality of two-dimensional projection images with respect to a coordinate grid adapted to the geometry of the transmitted X-rays; and generating a deconvolved three-dimensional image volume by applying a two-dimensional deconvolution to slices of the three-dimensional raw image volume, where the slices are adapted to the coordinate grid.

For example, the method may be performed during tomosynthesis and only a limited number of two-dimensional projection images may be acquired within a limited view angle range. The three-dimensional raw image volume may be generated by filtered back projection, which may generate artifacts (i.e. a non-singular point spread function) in the three-dimensional raw image volume. However, as the filtered back projection may be performed with respect to a geometry-matched coordinate grid, the artifacts of a point in a coordinate system aligned slice may only be situated in the slice and may be compensated by a two-dimensional deconvolution of the respective slice. A coordinate grid may be matched to the geometry of the X-ray imaging system, when its coordinate axes are aligned with the X-ray beam generated by the X-ray imaging system.

In general, a reconstruction of a three-dimensional image based on a geometry matched grid may be combined with a two-dimensional deconvolution to suppress artifacts, to enhance the z-resolution and/or to enhance the quality of the three-dimensional image. The deconvolved three-dimensional image volume may be used as input for further processing or further iterative reconstruction steps.

A further aspect of the invention relates to an X-ray device, which comprises an X-ray source and an X-ray detector that are adapted to acquire two-dimensional projection images of an object of interest, wherein the X-ray source and/or the X-ray detector are movable with respect to the object of interest for acquiring two-dimensional projection images with respect to different view angles; and a controller, which is adapted for performing the steps of the method as described in the above and in the following.

For example, the method and the X-ray device may be used in screening and diagnosis by mammographic tomosynthesis, i.e. the object of interest may be a breast.

It has to be understood that features of the method as described in the above and in the following may be features of the X-ray device as described in the above and in the following and vice versa.

These and other aspects of the invention will be apparent from and elucidated with reference to the embodiments described hereinafter.

### BRIEF DESCRIPTION OF THE DRAWINGS

Below, embodiments of the present invention are described in more detail with reference to the attached drawings.

FIG. 1 schematically shows an X-ray device according to an embodiment of the invention.

FIG. 2 shows a flow diagram for a method for processing image data of an X-ray device according to an embodiment of the invention.

FIG. 3 schematically shows a three-dimensional image processed during the method of FIG. 2.

FIG. 4A and 4B show slices through a three-dimensional image processed with a Cartesian coordinate grid.

FIG. 5 shows a slice through a three-dimensional image that has been back projected with a conical coordinate grid.

FIG. 6 shows a slice through a three-dimensional image deconvolved with a conical grid.

In principle, identical parts are provided with the same reference symbols in the figures.

### DETAILED DESCRIPTION OF EMBODIMENTS

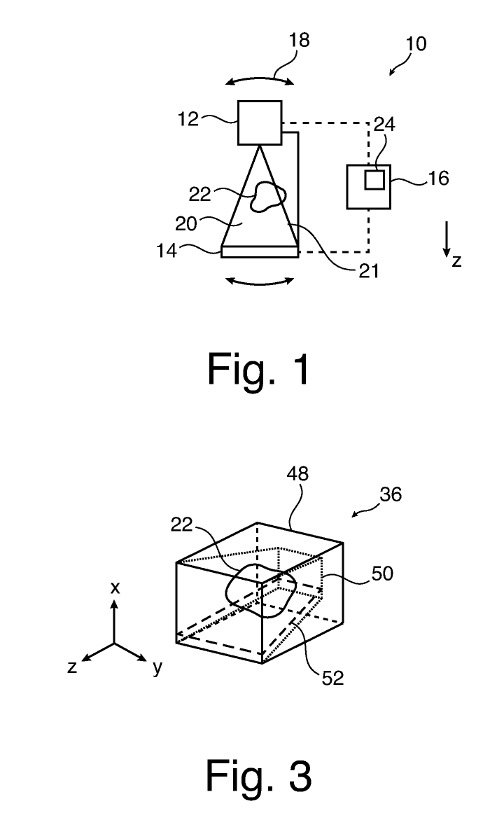

FIG. 1 schematically shows an X-ray device/system 10 comprising an X-ray tube/source 12 and an X-ray detector 14. The X-ray device may further comprise a controller 16 for controlling the X-ray device 10.

The X-ray tube 12 and the X-ray detector 14 may be mechanically interconnected and may be movable about an axis in a limited range 18, for example under the control of the controller 16, which may control the movement via a drive like an electrical motor. The X-ray tube 12 may generate X-rays 20 or an X-ray beam 20 in the form of a cone 21 that is transmitted through an object of interest 22. The detector 14 may acquire (raw) X-ray projection images of the object of interest 22 that may be further processed by the controller 16.

The X-ray device 10 may comprise a display device 24 for displaying images generated by the controller 16 based on the X-ray images acquired by the detector 14.

In particular, the X-ray device 10 may be a tomosynthesis device/system 10. Tomosynthesis is an imaging technique in which multiple X-ray images of the object of interest are taken from a discrete number of view angles. Tomosynthesis differs from computer tomography because the range 18 of view angles used is less than 360°, which is used in computer tomography. The cross-sectional X-ray images are then used to reconstruct three-dimensional images of the object of interest 22.

Because of the limited angle range 18, tomosynthesis may have a limited depth-resolution, in the direction of the X-rays, which is indicated as z-direction in FIG. 1.

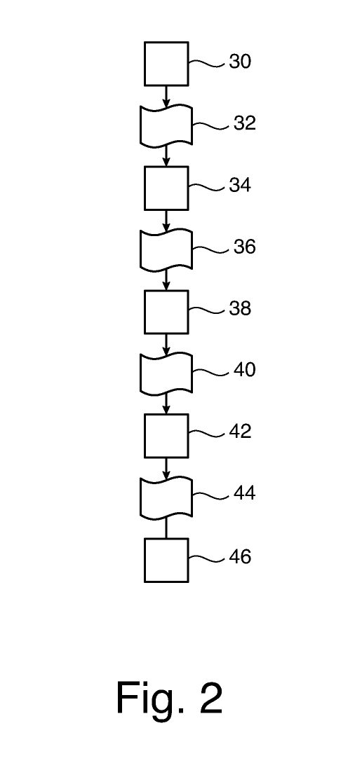

FIG. 2 shows a flow diagram for a method for processing image data of the X-ray device 10. The controller 16 of the X-ray device 10 may be adapted to perform the method. For example the controller 16 may comprise a processor and a memory, in which a computer program is stored, which when being executed on a processor is adapted for performing the steps of the method as described in the above and in the following. In general, such a program may be stored on a computer-readable medium.

A non-volatile computer-readable medium may be a floppy disk, a hard disk, an USB (Universal Serial Bus) storage device, a RAM (Random Access Memory), a ROM (Read Only Memory), an EPROM (Erasable Programmable Read Only Memory) or a FLASH memory. A volatile computer-readable medium may be a data communication network, e.g. the Internet, which allows downloading the computer program.

Turning back to FIG. 2, in step 30, a plurality of X-ray projection images 32 are acquired by the system of X-ray tube 12 and X-ray detector 14 and may be saved in a memory of the controller 16. The X-ray projection images 32 may be acquired in a limited range 18 and with a limited number of projection images 32.

According to an embodiment of the invention, the plurality of two-dimensional projection images 32 are acquired only in a limited angle range 18 of view angles, which may be, for example, less than 40°, less than 30° or less than 20°.

According to an embodiment of the invention, the plurality of two-dimensional projection images 32 comprises less than 30 projection images 32, for example less than 20 projection images 32 or less than 15 projection images 32.

It has to be noted that an X-ray image in general may be represented by digital image data that may be stored in a memory of the X-ray device 10 or the controller 16.

Usually, an X-ray image comprises an intensity value relating to the absorption of X-rays of the object 20 with respect to the X-rays. This may be either true for two-dimensional X-ray images (such as the projection images 32) as well as three-dimensional X-ray images (such as the images 36, 40, 44 mentioned below).

A two-dimensional X-ray image 32 may comprise pixels labelled with a two-dimensional coordinate and/or each pixel may be associated with an intensity value. In the end of step 30, the plurality of two-dimensional X-ray images 32 may be received and stored in the controller 16.

According to an embodiment of the invention, the method comprises the step of: receiving a plurality of two-dimensional projection images 32 from an object of interest 22, wherein the projection images have been acquired by transmitting X-rays 20 through the object of interest 20 with respect to different view angles.

In step 34, the controller 16 generates a three-dimensional X-ray raw image volume 36 from the plurality of two-dimensional X-ray projection images 32. For the generation of the three-dimensional image volume 36, a coordinate grid or coordinate system adapted to the geometry of the imaging system (the X-ray tube 12 and the X-ray detector 14) of the X-ray device 10 is used.

According to an embodiment of the invention, the method comprises the step of: generating a three-dimensional raw image volume 36 from the plurality of two-dimensional projection images 32 with respect to a coordinate grid adapted to the geometry of the transmitted X-rays 20.

FIG. 3 schematically shows a three-dimensional image volume 36 processed during the method of FIG. 2. In FIG. 3, an orthogonal (Cartesian) coordinate grid/system 48 and a geometry matched coordinate grid/system 50 are shown.

The coordinate grid 50 is adapted to the cone 21 of X-rays 20 of the X-ray device 10. With growing z-coordinate, the unit vectors of the x- and y-coordinate are growing accordingly.

According to an embodiment of the invention, the coordinate grid 50 defines a cone with respect to an orthogonal grid.

The angle of the cone defined by the coordinate grid 50 may be the same as the angle of the cone 21 of X-rays generated by the X-ray tube/source 12. In other words, the coordinate lines of constant x and y may run along lines that match to X-rays transmitted through the object of interest 22.

According to an embodiment of the invention, the X-rays 20 are generated by a point source 12 and are transmitted through the object of interest 22 via a cone beam 21 and the coordinate grid has coordinate lines running along the cone beam.

In general, a three-dimensional X-ray image comprises voxels labelled with a three-dimensional coordinate, which in the present case need not be based on a Cartesian coordinate system, but a coordinate system adapted to the geometry of the X-ray device, for example a coordinate system, where the unit vector for x and y linearly increases with increasing z. Each voxel usually may comprise an intensity value relating to the absorption of X-rays of the object 20 with respect to the X-rays.

For the generation of the three-dimensional X-ray image volume 36, filtered back projection or even more sophisticated iterative methods may be used. Filtered back projection is well known from computer tomography. However, in computer tomography, two-dimensional images acquired in view angles around the whole 360° of the object of interest are used.

According to an embodiment of the invention, the three-dimensional raw image volume 36 is generated by filtered back projection of the two-dimensional projection images 32 with respect to the coordinate grid 50.

Compared to other techniques such as shift-and-add (SAA), filtered back projections usually result in sharper point spread functions (PSF). A point spread function may describe the response of the imaging system of the X-ray device 10 to a point-like object of interest 22, i.e. the image that is generated by the X-ray device from a point-like object of interest 22.

Filtered back projection and an iterative reconstruction (see step 40 below) is usually performed on a Cartesian coordinate grid 48.

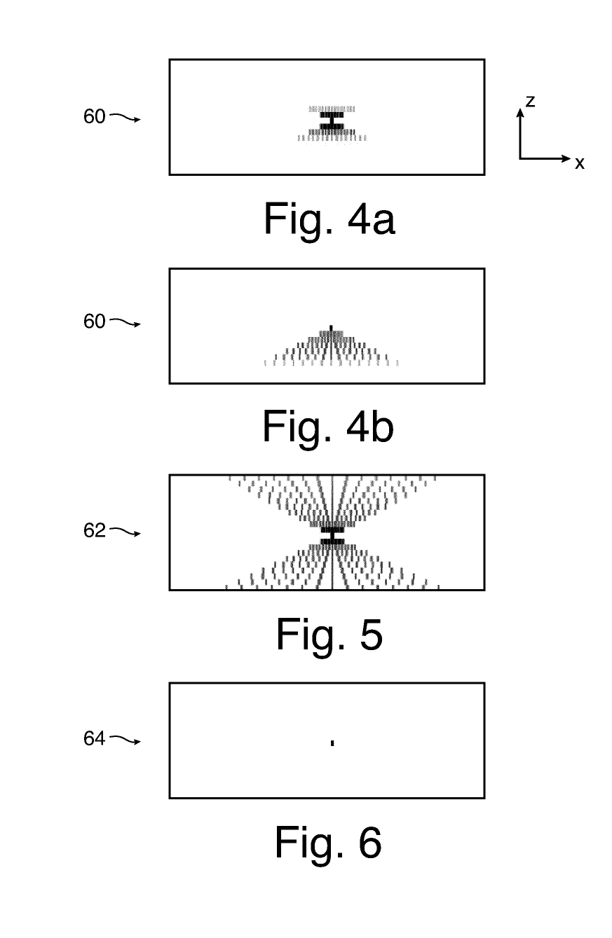

In this case, the point spread function is however not aligned with the Cartesian coordinate grid 48, as shown in FIG. 4A and 4B.

FIG. 4A and 4B (as well as FIGS. 5 and 6) show slices through a three-dimensional image parallel to the z-direction (where z defined as the principal direction of the X-rays). For example, the y-coordinate may be kept fixed to produce such slices. All FIG. 4A to 6 show examples with 15 projections, i.e. with 15 two-dimensional X-ray projection images 32.

FIG. 4A and 4B show the point spread function 60 of a filtered back projection with respect to a Cartesian coordinate grid 48. The reconstructed three-dimensional image of a very small object (the point spread function extends not only in the central slice (FIG. 4A) but also into adjacent slices (FIG. 4B).

FIG. 5 shows the point spread function 62 of a filtered back projection of a point-like object with respect to the coordinate grid 50 that is matched to the geometry of the X-ray device. FIG. 5 shows a slice, which comprises the point-like object. The complete point spread function 62 is situated in this slice. Adapting the grid geometry to the beam geometry (e.g. a conical grid) may allow for concentrating the point spread function 62 in a single slice.

Additionally, with the geometry matched grid 50 the point spread function may be spatially more constant along the readout direction, i.e. the z-direction. The point spread function 62 may become planar but its z-resolution may not improve.

The point spread function 62 shown in FIG. 5 may be seen as artifacts of filtered back projection in the three-dimensional image volume 36.

According to an embodiment of the invention, the artifacts and/or the point spread function are fan-shaped.

In step 38, a deconvolved three-dimensional image 40 is generated from the back projected three-dimensional image volume 36.

It is possible to perform the deconvolution in three dimensions. However, deconvolution in three dimensions may be computationally demanding, prone to noise and artifacts due to a large under-determined system of equations and hence hardly feasible in practice.

However, with the method, the deconvolution is performed only in two dimensions. A general problem of deconvolution in tomosynthesis (and in computer tomography in general) may be that the point spread function 60 is spatially dependent. Hence, frequency-domain-based approaches (e.g. Wiener deconvolution) may be problematic. Instead, image-domain-based deconvolution might be required.

With the method, the deconvolution of filtered back projected reconstructed tomosynthesis images is possible by operating slice-by-slice on a geometry-matched grid 50. This approach may take advantage of the much sharper point spread function 62 provided by filtered back projection and may operate in two dimensions only. With the method, the conditions of the numerical problem may be significantly eased.

As indicated in FIG. 2, the filtered back projection and the deconvolution are performed with respect to the coordinate grid 48 aligned with the geometry of the cone beam 21. In such a geometry, the point spread function 62 may be almost perfectly aligned with the slices of the coordinate grid 50 such that a two-dimensional deconvolution may be applied to recover the full three-dimensional X-ray image 40.

For example, the two-dimensional deconvolution may be performed in a slice 52, which is parallel to the X-rays of the beam 20. This is the case, for example, when one of the coordinates x or y is kept constant in the slice 52.

According to an embodiment of the invention, the slices 52 of the three-dimensional raw image volume 36 have a constant coordinate value with respect to the coordinate grid 50.

According to an embodiment of the invention, the method comprises the step of: generating a deconvolved three-dimensional image 40 by applying a two-dimensional deconvolution to slices 52 of the three-dimensional raw image volume 36, which slices 52 are adapted to the coordinate grid 50.

For performing the deconvolution, every slice 52 may be deconvolved with a kernel function that matches the point spread function and/or artifacts 62 produced by the filtered back projection. The kernel function may be spatially varying.

According to an embodiment of the invention, each slice 52 of the three-dimensional raw image volume 36 is deconvolved with a two-dimensional kernel function.

In principal, the kernel function may be equal to the point spread function 62. After deconvolution with the kernel function, the point spread function 62 is ideally mapped to a point function 64 or point-like function 64 as shown in FIG. 6. In other words, the deconvolution may be seen as the inverse transformation of the transformation that projects a point-like object into the point spread function 62.

According to an embodiment of the invention, the kernel function is adapted for mapping artifacts in the slice 52, which are generated from a point-like part of the object of interest 22 during reconstruction of the three-dimensional raw image volume 36, back to a point in the slice 52 corresponding to the point-like part.

Summarized, with the method, geometric information about the X-ray device 10, and more precisely the point spread function 62 is used to recover the full three-dimensional image 40 by deconvolution. The deconvolution may be performed on a coordinate grid 50 (for example a conical grid) to reduce the deconvolution to a two-dimensional problem. The two-dimensional deconvolution may be applied to three-dimensional tomosynthesis images, which have been reconstructed via filtered back-projection, taking advantage of their sharper point spread function. Overall, the method may facilitate significantly improved depth resolution in tomosynthesis and may reduce artifacts, especially when the angular view range is small. The improved z-resolution provided by the method may be seen in FIG. 6 compared to FIG. 4A.

In optional step 40, the three-dimensional image 36 obtained after the deconvolution may be used as a start image for iterative reconstruction. In other words, an iteratively reconstructed three-dimensional image 44 may be generated from the deconvolved three-dimensional image 36.

According to an embodiment of the invention, the method comprises the step of: iteratively reconstructing the deconvolved three-dimensional image 40.

During an iterative reconstruction, the three-dimensional image 40 may be forward projected to two-dimensional images and compared with the two-dimensional image 32. From the differences, errors in the generation of the three-dimensional image 36 during step 34 and/or the deconvolution during step 38 may be determined and corrected. The forward projection and the comparison may be performed several times on the newly generated corrected three-dimensional image 44, i.e. iteratively.

An iterative reconstruction may be especially advantageous as the improvement of the depth-resolution may lie within the null-space of the iterative reconstruction problem and is hence maintained through the iterations. Moreover, noise and deconvolution artifacts may be improved by an iterative approach.

In step 46, slices of the three-dimensional image 40, 44 may be displayed on the display device 24. Such a slice, which, for example may be orthogonal to the z-direction may be seen as a two-dimensional image that is reconstructed from the three-dimensional image 40 or 44.

According to an embodiment of the invention, the method comprises the step of: generating a reconstructed two-dimensional image based on a slice through the deconvolved or reconstructed three-dimensional image 40.

According to an embodiment of the invention, the method comprises the step of: displaying the reconstructed two-dimensional image on a display device 24.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practising the claimed invention, from a study of the drawings, the disclosure, and the appended claims. In the claims, the word “comprising” does not exclude other elements or steps, and the indefinite article “a” or “an” does not exclude a plurality. A single processor or controller or other unit may fulfil the functions of several items recited in the claims. The mere fact that certain measures are recited in mutually different dependent claims does not indicate that a combination of these measures cannot be used to advantage. Any reference signs in the claims should not be construed as limiting the scope.

1. A method for processing image data of an X-ray device, the method comprising the steps of:
receiving a plurality of two-dimensional projection images from an object of interest, wherein the projection images have been acquired by transmitting X-rays through the object of interest with respect to different view angles;
generating a three-dimensional raw image volume from the plurality of two-dimensional projection images with respect to a coordinate grid adapted to the geometry of the transmitted X-rays;
generating a deconvolved three-dimensional image by applying a two-dimensional deconvolution to slices the three-dimensional raw image volume which slices are adapted to the coordinate grid.

2. The method of claim 1,
wherein the coordinate grid defines a cone with respect to an orthogonal grid.

3. The method of claim 1
wherein the X-rays are generated by a point source and are transmitted through the object of interest via a cone beam;
wherein the coordinate grid, has coordinate lines running along the cone beam.

4. The method of claim 1,
wherein the three-dimensional raw image volume is generated by filtered back projection of the two-dimensional projection images with respect to the coordinate grid.

5. The method of claim 1,
wherein the slices of the three-dimensional raw image volume have a constant coordinate value with respect to the coordinate grid.

6. The method of claim 1,
wherein each slice of the three-dimensional raw image volume is deconvolved with a two-dimensional kernel function.

7. The method of claim 1,
wherein the kernel function is adapted for mapping artifacts in the slice, which are generated from a point-like part of the object of interest, during reconstruction of the three-dimensional raw image volume, back to a point in the slice corresponding to the point-like part.

8. The method of claim 7,
wherein the artifacts are fan-shaped.

9. The method of claim 1, further comprising the step of:
performing further iteratively reconstruction using the deconvolved three-dimensional image as a start image.

10. The method of one of the preceding claims,
wherein the plurality of two-dimensional projection images are acquired only in a limited angle range of view angles.

11. The method of claim 1,
wherein the plurality of two-dimensional projection images comprises less than 30 images.

12. The method of claim 1, further comprising the step of:
generating a reconstructed two-dimensional image based on a slice through the deconvolved three-dimensional image;
displaying the reconstructed two-dimensional image on a display device.

13. A computer program for processing image data of an X-ray device, which when executed on a processor is adapted for performing the steps of the method of claim 1.

14. A computer-readable medium, on which a computer program according to claim 13 is stored.

15. An X-ray device , comprising:
an X-ray source, and an X-ray detector that are adapted to acquire two-dimensional projection images of an object of interest, wherein the X-ray source and/or the X-ray detector are movable with respect to the object of interest for acquiring two-dimensional projection images with respect to different view angles; and
a controller, which is adapted for performing the steps of the method of claim 1.
